(19) United States
(12) Patent Application Publication
(10) Pub. No.: US 2014/0065245 A1
(43) Pub. Date: Mar. 6, 2014

(54) METHOD FOR PREPARING DIETARY SUPPLEMENTS

(71) Applicant: Pai-Ying Hsu, Taipei (TW)

(72) Inventor: Pai-Ying Hsu, Taipei (TW)

(73) Assignee: NATURE'S SELECT INTERNATIONAL INC., CARSON CITY, NV (US)

(21) Appl. No.: 13/598,658

(22) Filed: Aug. 30, 2012

(51) Int. Cl. A61K 31/70; A61K 38/39; A61P 19/00; A61K 33/08; A61K 36/00; A61K 36/328; A61K 33/06; A61K 33/10

(52) U.S. Cl. 424/682; 514/62; 514/17.2; 424/687; 424/692; 424/725; 424/748

(57) ABSTRACT

A novel dietary supplement and methods for manufacturing the said dietary supplement are disclosed for promoting healthy cartilages, connective tissues and bones. The dietary supplement of the present invention comprises glucosamine hydrochloride, collagen, and other dietary ingredients. Some ingredients of the dietary supplement of the present invention help to maintain the functions of cartilages, connective tissues and bones. Some ingredients of the dietary supplement of the present invention also exhibit anti-inflammatory properties. Overall, these ingredients of the dietary supplement are beneficial to the health of human and other animals.

### BACKGROUND

Aspects of the present invention relate generally to the field of dietary supplements, and more specifically, to the preparing of a dietary supplement that promotes healthy cartilages and connective tissues.

Arthritis is an inflammatory disorder affecting the cartilages and bones of the joint systems. Osteoarthritis is a very common form of arthritis. Causes of osteoarthritis include genetic, mechanical and metabolic problems. When a person is infected with osteoarthritis, the person's cartilages may degenerate and the person's synovial fluid may decrease. Then, without the cushioning effect of the cartilages and the lubrication offered by the synovial fluid, the bones in a joint may start rubbing against each other. Thus, when a person moves, the friction in a joint will cause pain, thereby limiting a person's mobility. In addition, if the friction between the bones of a joint continues, it will cause progressive inflammation, swelling and further degradation to the cartilages.

Osteoarthritis affects approximately one third of adults aged 65 years and above. Joint systems that are more susceptible to Osteoarthritis include joint systems within hands, spine, knees, and hips. Common symptoms of osteoarthritis include joint pain, stiffness, swelling, and lost of joint functions.

Treatments for osteoarthritis involve pain control and improvement in joint functions.

Several clinical studies have shown glucosamine as a safe ingredient for treating osteoarthritis, wherein the glucosamine is shown to be capable of improving the function of the joint systems and decreasing joint pain.

Accordingly, there is a need in the art for a dietary supplement that utilizes the benefits of glucosamine and other dietary ingredients to promote healthy cartilages and bones of the joint systems.

### SUMMARY

The present invention discloses a method to prepare a dietary supplement that promotes healthy cartilages and bones of the joint systems. In a method according to an embodiment of the present invention, the temperature of a solvent is first adjusted to a first temperature. Then, a first ingredient group is added to the solvent to form, after a filtration process, a filtered first solution. Next, the filtered first solution is adjusted to a second temperature and is subsequently mixed with a second ingredient group to form a second solution. Finally, the second solution is cooled to a third temperature and distributed into containers.

According to an embodiment of the present invention, the first ingredient group comprises Rhizoma dioscoreae Nipponicae and Clematis chinensis, and the second ingredient group comprises glucosamine hydrochloride, calcium supplementation, collagen type II, and vitamin D.

### BRIEF DESCRIPTION OF THE DRAWINGS

The described embodiments of the present invention will be apparent through examination of the following detailed description in conjunction with the accompanying drawings, in which:

FIG. 1 is a schematic drawing of a knee joint showing the anatomy of a human's knee joint system.

FIG. 2 is a simplified flow diagram illustrating a method to prepare the dietary supplement according to an embodiment of the present invention.

### DETAILED DESCRIPTION

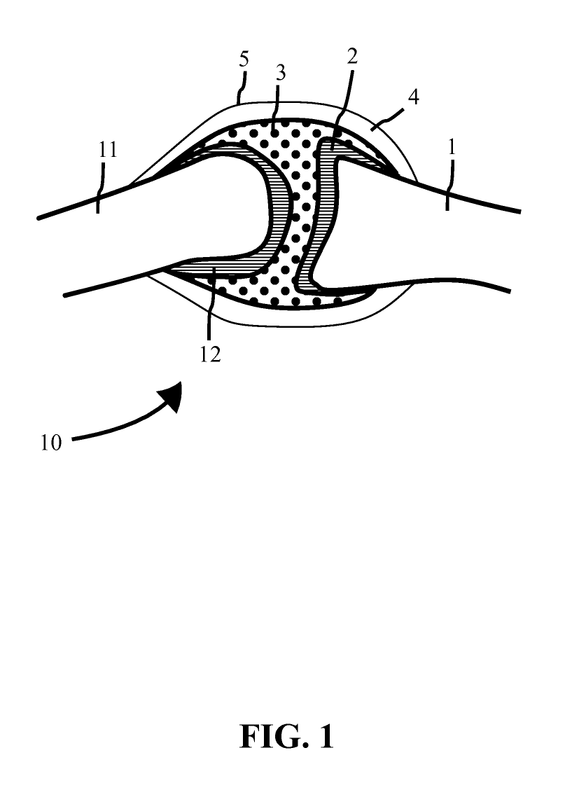

FIG. 1 is a schematic drawing of a knee joint showing the anatomy of a human's knee joint system 10. Although the present invention may improve the health of a human's knee joint systems, the present invention is not so limited. As will be apparent to a person of ordinary skill in the art, the present invention may also improve the health of elbow joint systems, wrist joint systems, finger joint systems, and other joint systems of a human body. In addition, the present invention may also apply to other animals, such as dogs, cats, and horses. In FIG. 1, the knee joint system 10 includes the bone 1 and bone 11, which are components of a skeletal system that provides a supporting framework for a human body. As shown, one end of the bone 1 is encased in the cartilage 2 and one end of the bone 11 is encased in the cartilage 12. The cartilage 2 and cartilage 12 are flexible connective tissues that function as shock absorbers to allow a human body to sustain impacts. The synovial fluid 3 between the cartilage 2 and cartilage 12 serves as a lubricant in the knee joint system 10. The cushioning effect from the combination of the cartilage 2, cartilage 12 and synovial fluid 3 allows the bone 1 and bone 11 of the joint system 10 to move freely without friction. The synovial membrane 4 is responsible for the production of the synovial fluid 3. The synovial membrane 4 and joint capsule 5 act as protective layers for the ends of the bone 1, the bone 11, the cartilage 2, the cartilage 12, and the synovial fluid 3 in the joint system 10.

According to an embodiment of the present invention, an ingredient for the dietary supplement may be glucosamine. Glucosamine is an amino monosaccharide found in humans. Glucosamine may be the precursor for the synthesis of glycosaminoglycans and the formation of proteoglycans by chondrocytes. Since proteoglycans are vital components to form the cartilages, glucosamine may serve as a fundamental element for cartilage regeneration. In addition, glucosamine may also help thickening the synovial fluids and may alleviate the pain associated with osteoarthritis. Consequently, the intake of glucosamine may enhance the function of the cartilages, thereby promoting joint health and decreasing joint pain associated with osteoarthritis. Glucosamine may be formulated as a dietary ingredient in the form of glucosamine hydrochloride or glucosamine sulfate.

Another ingredient for the dietary supplement according to an embodiment of the present invention may be collagen. There are various types of collagen in the human body, wherein collagen type II is found in human cartilages. Collagen type II may be efficacious in the treatment of osteoarthritis and rheumatoid arthritis. Thus, collagen type II may be an ingredient to the dietary supplement according to an embodiment of the present invention.

Another ingredient for the dietary supplement according to an embodiment of the present invention may be chondroitin. Chondroitin is a form of glycosaminoglycan, which is an element to form the cartilages. As a dietary ingredient, Chondroitin may be extracted from shark or bovine cartilages in the form of chondroitin sulfate, which may be an effective treatment for knee osteoarthritis.

Another ingredient for the dietary supplement according to an embodiment of the present invention may be calcium. Different types of calcium salt may be used. Different calcium salts may yield different amounts of calcium. For example, calcium glucanate may yield 9-10% calcium, calcium citrate may yield 21% calcium, calcium lactate may yield 13% calcium, and calcium carbonate may yield 40-50% calcium.

Another ingredient for the dietary supplement according to an embodiment of the present invention may be magnesium. In a human body, magnesium resides mostly in the bone structures. Magnesium is essential for maintaining a healthy skeletal structure and for enhancing bone densities. Magnesium may be formulated as a dietary ingredient in the form of magnesium oxide.

Another ingredient for the dietary supplement according to an embodiment of the present invention may be yucca. In powder form, Yucca may be extracted from the bark of Mohave Yucca, a desert plant. Yucca may reduce joint pain with its anti-inflammatory property.

Another ingredient for the dietary supplement according to an embodiment of the present invention may be zinc. Zinc may help to regulate the production of collagen and other connective proteins of the joint system. Zinc may be formulated as a dietary ingredient in the form of zinc gluconate.

Another ingredient for the dietary supplement according to an embodiment of the present invention may be Curcuma longa. Curcuma longa may be in a yellow powdery form extracted from the root of a turmeric perennial shrub. Curcumin, a chemical present in Curcuma longa, may have anti-inflammatory property and may help to alleviate the pain associated with osteoarthritis.

Another ingredient for the dietary supplement according to an embodiment of the present invention may be Manganese. Manganese is a trace mineral that may be found in the bone structures, and may assist the process of bone formation. Manganese may also heighten the effectiveness of glucosamine to the joint system. Manganese may be taken in the form of manganese sulfate as a dietary ingredient.

Another ingredient for the dietary supplement according to an embodiment of the present invention may be copper, whose antioxidative property may reduce the inflammation associated with osteoarthritis. Copper may be taken in the form of copper gluconate as a dietary ingredient.

Another ingredient for the dietary supplement according to an embodiment of the present invention may be bromelain. Bromelain may be extracted from pineapple plants, and may have analgesic and anti-inflammatory properties. Bromelain may also help to reduce the inflammation associated with osteoarthritis, and may also enhance the efficacy of glucosamine.

Another ingredient for the dietary supplement according to an embodiment of the present invention may be vitamin D. Vitamin D may facilitate the absorption of the calcium supplementation by the human body. Vitamin D3 may be an effective form of vitamin D. The intake of vitamin D3 with the calcium supplementation may improve bone densities.

Another ingredient for the dietary supplement according to an embodiment of the present invention may be an herbal or plant material. There are several herbal or plant materials that have been shown to be beneficial to human joint systems. For example, the combination of Rhizoma dioscoreae Nipponicae, Clematis chinensis, frankincense and myrrh may exhibit anti-inflammatory properties effective in decreasing joint swelling, alleviating joint pain, and improving a person's mobility. Thus, by adding these herbal or plant materials to the glucosamine supplements (such as glucosamine hydrochloride or glucosamine sulfate), the combination may exhibit a greater benefit to human joint systems.

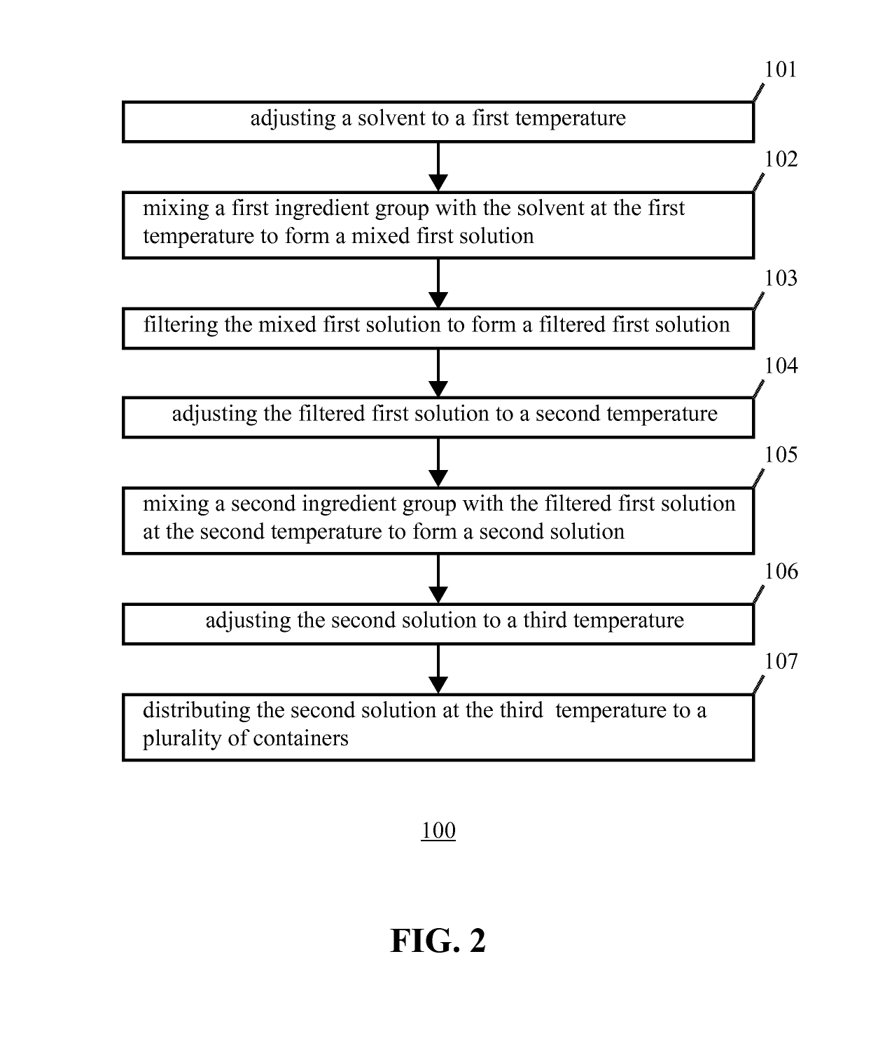

FIG. 2 is a simplified flow diagram illustrating a method to prepare the dietary supplement according to an embodiment of the present invention.

As shown, in step 101, a solvent is heated or cooled to a first temperature. A solvent may be water, distilled water, water from reverse osmosis, or other sterile solvent that is compatible with the dietary ingredients and safe for human consumption.

Then, at step 102, a first ingredient group is mixed with the solvent to form a mixed first solution. For example, the first ingredient group may comprise herbal or plant materials. A common method to extract the active ingredients from herbal or plant materials is the decoction method. Thus, an embodiment of the present invention is to brew the herbal or plant materials for a period of time, which may be optimized for the extraction of the active ingredients within the herbal or plant materials. Consequently, the first temperature may be set at the recommended decocting temperature associated with the herbal or plant materials. This decocting temperature may be the boiling point of water, which is approximately between 95 degrees Celsius and 105 degrees Celsius.

According to an embodiment of the present invention, the first ingredient group may comprise Rhizoma dioscoreae Nipponicae, Clematis chinensis, frankincense, myrrh, or combinations thereof. According to an embodiment of the present invention, the relative weight ratio among these materials may be Rhizoma dioscoreae Nipponicae (40%), Clematis chinensis (40%), frankincense (10%), and myrrh (10%). These materials may then be placed in boiling water for approximately 45 minutes to extract their active ingredients. The first ingredient group may also include other herbal or plant ingredients beneficial to human joint systems.

Once the extraction of the active ingredients in the first ingredient group is completed, at step 103, the mixed first solution may be filtered to remove the remaining insoluble substances and may become a filtered first solution. The filtration process may be accomplished by physically blocking the insoluble substances while letting others through. A person of ordinary skill in the art would appreciate that there are many filtration methods that can be used to separate the insoluble substances, such as by using a filter paper or a surface filter.

Thereafter, at step 104, the filtered first solution may be adjusted to a second temperature, and, at step 105, be mixed with a second ingredient group. Alternatively, a personal of ordinary skill in the art would appreciate that the mixed first solution may first be cooled to the second temperature, and then be filtered and mixed with the second ingredient group.

According to an embodiment of the present invention, the second ingredient group may comprise glucosamine hydrochloride, calcium supplement, collagen type II, vitamin D3 or combinations thereof. In one example, the relative weight ratio among the glucosamine hydrochloride, the collagen type II, the calcium supplement, and the vitamin D is about 40%:55%:4%:1%.

In addition, according to an embodiment of the present invention, the second ingredient group may comprise glucosamine hydrochloride, collagen type II, chondroitin, calcium glucanate, calcium citrate, calcium lactate, calcium carbonate, magnesium oxide, yucca, zinc gluconate, curcuma longa, manganese sulfate, copper gluconate, bromelain and vitamin D3, or combinations thereof. Preferably, the relative weight among these materials may be approximately glucosamine hydrochloride (28.23%), collagen type II (42.35%), chondroitin (16.94%), calcium glucanate (1.76%), calcium citrate (1.76%), calcium lactate (1.76%), calcium carbonate (1.76%), magnesium oxide (1.69%), yucca (0.03%), zinc gluconate (0.07%), curcuma longa (0.04%), manganese sulfate (0.04%), copper gluconate (0.01%), bromelain (0.001%), and vitamin D3 (0.001%).

According to an embodiment of the present invention, the second temperature may be in an approximate range that readily dissolves all the ingredients in the second ingredient group. For example, if the second ingredient group comprises glucosamine hydrochloride, the second temperate may be approximately between 60 degrees and 80 degrees Celsius. At the second temperature, materials from the second ingredient group may dissolve in the filtered first solution to form a second solution. To speed up the dissolution process, a blending process may be utilized.

Finally, at step 106, the method 100 comprises adjusting the temperature of the second solution to a third temperature. The third temperature may allow the second solution to be injected, at step 107, into a container without deforming the container. For example, a third temperature may range from 50 degrees to 60 degrees Celsius. The containers may be made of plastic, glass, or other materials.

The dietary supplement produced by method 100 is in a liquid form, but the present invention is not so limited. Instead, a person of ordinary skill in the art would appreciate that the present invention also applies to dietary supplement in the forms of pills, capsules, and tablets. For example, after step 106, the second solution may be cooled, submitted to filtration process, and dried in a vacuum oven, so that the second solution may be transformed into a solid form. The solid form of the dietary supplement may be further processed, such as being divided in equal proportions and enclosed in capsules.

While the invention has been described in detail above, a person of ordinary skill in the art shall recognize that there are apparent variations within the scope and spirit of the invention. Therefore, the scope of the present invention should be considered as only limited by the scope of the appended claims.

What is claimed is:

1. A method for preparing a dietary supplement comprising:
adjusting a solvent to be at a first temperature;
mixing a first ingredient group with the solvent at the first temperature to form a mixed first solution;
filtering the mixed first solution to form a filtered first solution;
adjusting the filtered first solution to be at a second temperature;
mixing a second ingredient group with the filtered first solution at the second temperature to form a second solution;
adjusting the second solution to be at a third temperature; and
distributing the second solution at the third temperature to a plurality of containers.

2. The method of claim 1 wherein said second ingredient group comprises glucosamine hydrochloride.

3. The method of claim 1 wherein said second ingredient group comprises glucosamine sulfate.

4. The method of claim 2 wherein said second ingredient group further comprises a calcium supplement, collagen type II, and vitamin D.

5. The method of claim 4 wherein a weight ratio among the glucosamine hydrochloride, the collagen type II, the calcium supplement, and the vitamin D is about 40:55:4:1.

6. The method of claim 1 wherein said second ingredient group comprises glucosamine hydrochloride, collagen type II, chondroitin, calcium glucanate, calcium citrate, calcium lactate, calcium carbonate, magnesium oxide, yucca, zinc gluconate, curcuma longa, manganese sulfate, copper gluconate, bromelain, and vitamin D3.

7. The method of claim 4 wherein the second temperature is approximately between 60 degrees Celsius and 80 degrees Celsius.

8. The method of claim 1 wherein the first ingredient group comprises an herbal material.

9. The method of claim 1 wherein the first ingredient group comprises a plant material.

10. The method of claim 8 wherein the first temperature corresponds to a recommended decocting temperature associated with the herbal material.

11. The method of claim 1 wherein the first ingredient group comprises Rhizoma dioscoreae Nipponicae.

12. The method of claim 1 wherein the first ingredient group comprises Rhizoma dioscoreae Nipponicae and Clematis chinensis in approximately equal weight.

13. The method of claim 12 wherein the first ingredient group further comprises frankincense and myrrh.

14. The method of claim 8 wherein the first temperature is approximately between 95 degrees Celsius and 105 degrees Celsius.

15. The method of claim 14 wherein the herbal material is brewed at the first temperature for over 30 minutes.

16. The method of claim 1 wherein the first ingredient group comprises Rhizoma dioscoreae Nipponicae and Clematis chinensis, and the second ingredient group comprises glucosamine hydrochloride, a calcium supplement, collagen type II, and vitamin D.

17. The method of claim 16 wherein the first temperature is at about 100 degrees Celsius, the second temperature is approximately between 60 degrees Celsius and 80 degrees Celsius, and the third temperature is approximately between 50 degrees Celsius and 60 degrees Celsius.

18. The method of claim 17 wherein the first ingredient group is brewed at the first temperature for over 30 minutes.

19. The method of claim 18 wherein the second ingredient group further comprises magnesium oxide, yucca, zinc gluconate, curcuma longa, manganese sulfate, copper gluconate, and bromelain.

20. A dietary supplement manufactured by the method of claim 1.
